(19) United States
(12) Patent Application Publication
(10) Pub. No.: US 2019/0365547 A1
(43) Pub. Date: Dec. 5, 2019

(54) Method and apparatus for fixating endovascular prostheses

(71) Applicant: Amsel Medical Corporation, Cambridge, MA (US)

(72) Inventors: Arnold Miller, Cambridge, MA (US); Raanan Miller, Cambridge, MA (US); Nir Lilach, Kfar Yehoshua (IL)

(21) Appl. No.: 16/383,222

(22) Filed: Apr. 12, 2019

Related U.S. Application Data
(60) Provisional application No. 62/656,786, filed on Apr. 12, 2018.

(51) Int. Cl. A61F 2/848; A61F 2/07

(52) U.S. Cl. CPC A61F 2/848; A61F 2002/9665; A61F 2/07

(57) ABSTRACT

Method and apparatus are provided for endoluminally fixating and endoluminal prosthesis to a vessel wall to preclude migration of the prosthesis.

### FIELD

The invention relates to methods and devices for fixing the position of an endovascular prosthesis within the lumen of a hollow anatomical structure, such as a blood vessel.

### BACKGROUND

Prostheses, such as endovascular vascular stents and stent-grafts are widely used to treat numerous medical conditions, for example, to maintain patency of a compromised or obstructed blood vessel by placing the stent or stent-graft within the lumen of the vessel. The placement and fixation of such prostheses in the targeted position within the lumen of the vessel is important, as is the ability of the prosthesis to remain in that position. To that end, such prostheses, which typically include a tubular, wire-like framework, are expanded against the inner wall of the vessel to firmly engage the vessel to maintain the prosthesis in place. In the case of a stent-graft, a tubular graft in the form of a fabric (e.g., Dacron) or unwoven material (e.g., expanded PTFE) is attached to and is supported by the framework and, when placed within the lumen of the vessel, functions as a prosthetic portion of the vessel . In some instances, however, even if the prosthesis is placed in the proper location, it may tend to migrate downstream, for example, as a result of exposure to repeated pulsatile blood. The risk of migration is particularly problematic when the prosthesis is used to treat an arterial aneurysm and, especially, an aneurysm of the aorta, such as an abdominal aortic aneurysm (AAA). AAA results from a weakening of a portion of the wall of the aorta so that the diameter of the aorta at that location increases in response to the blood pressure. Should the weakened wall of an AAA burst, the high rate of internal hemorrhage is life threatening. Invasive surgical repair of an AAA is a delicate procedure and has a relatively high mortality. Consequently, less invasive techniques and devices have been developed to enable clinicians to access the aorta intraluminally so as to place a stent-graft within the aorta to effectively line the lumen of the aorta and relieve the aneurysmal region. The stent-graft should be configured to engage and seal against an unweakened portion of the aorta at least above the weakened region of the aneurysm and also may be secured below the aneurysm. Such stent-grafts typically are delivered and deployed intraluminally by a percutaneous delivery catheter that can be advanced through the patient's vasculature to the site of the aneurysm where the stent-graft is deployed and expanded into engagement with the inner luminal surface of the vessel.

In order to reduce the risk of stent-graft migration, such stent-grafts may be provided with additional means to fix the position of the deployed stent-graft. Thus, the framework of some stent-grafts have been provided with barbs or hooks as part of the framework of the stent adapted to dig into the vessel wall. Other fixation devices have included, among others, corkscrew-like fasteners with a sharp tip adapted to pierce the graft and the aorta wall to secure the position of the prosthesis. Such tissue-piercing devices have been reported (e.g., U.S. Patent Pub. 2009/0270976) to work loose over time and that may result in stent-graft migration and/or blood leaking into the space between the graft and the vessel wall or other risks to the patient. It would be desirable to provide an improved means to fix the position of such tubular prosthesis.

### SUMMARY

The invention provides a catheter-deliverable fastener that can be advanced percutaneously and intraluminally into the aorta or other vessel and be deployed to secure the prosthesis to the vessel wall. The fastener is contained in a hollow needle that is deliverable by the catheter to the target area. The needle then is advanced out of the catheter and is directed partly in a radial direction so that the needle pierces the stent-graft and the wall of the vessel to transfix the graft and vessel wall and locate the tip of the needle just outside of the vessel. The fastener may be of two-piece construction, having a distal component and a proximal component, that are containable in tandem in a low profile in the lumen of the needle during delivery. Each of the proximal and distal components can expand to larger diameter when ejected from the needle. The needle is provided with a limit stop on its outer surface that is adapted to engage the inner surface of the stent-graft so as to prevent the sharp tip of the needle from projecting much beyond the outer surface of the vessel wall, to avoid potentially damaging surrounding tissue. With the needle tip just beyond the outside of the vessel wall, the distal portion of the fastener is deployed, followed by retraction of the needle into the lumen of the stent-graft, and the proximal portion of the fastener then is deployed. Both portions of the fastener self-deploy to their expanded configuration upon release from the needle with an intermediate portion of the fastener passing through the needle-formed puncture in the stent-graft and the vessel wall, transfixing and securing both. The deployment apparatus then can be withdrawn. One or more of such fasteners can be placed, as desired.

### DRAWINGS

The various objects and advantages of the invention will be appreciated more fully from the following further description, with reference to the accompanying drawings in which:

FIG. 1 is a diagrammatic illustration of a portion of an aorta in which an aneurysm has formed and in which a stent-graft has been placed through and beyond the diseased, weakened region;

FIG. 2 is an exploded illustration of an exemplary two-piece fastener as may be used in the practice of the invention with its components in a relaxed, unstressed configuration;

FIGS. 3-7 are sequential illustrations of the manner in which the two-piece fastener of FIG. 2 is deployed;

FIG. 8 is an enlarged illustration of the distal end of the delivery catheter with the needle extended and in a position after deployment of the distal of the fastener components;

FIG. 8A is a further enlarged illustration of the portion of the needle on which the limit stop is formed with the stop in its active position to limit the extent that the needle can extend beyond the vessel pierced by the needle; and

FIG. 9 is an illustration similar to FIG. 8 showing the device within the lumen of the vessel and in readiness to deploy the distal of the fastener components.

### ILLUSTRATIVE EMBODIMENT

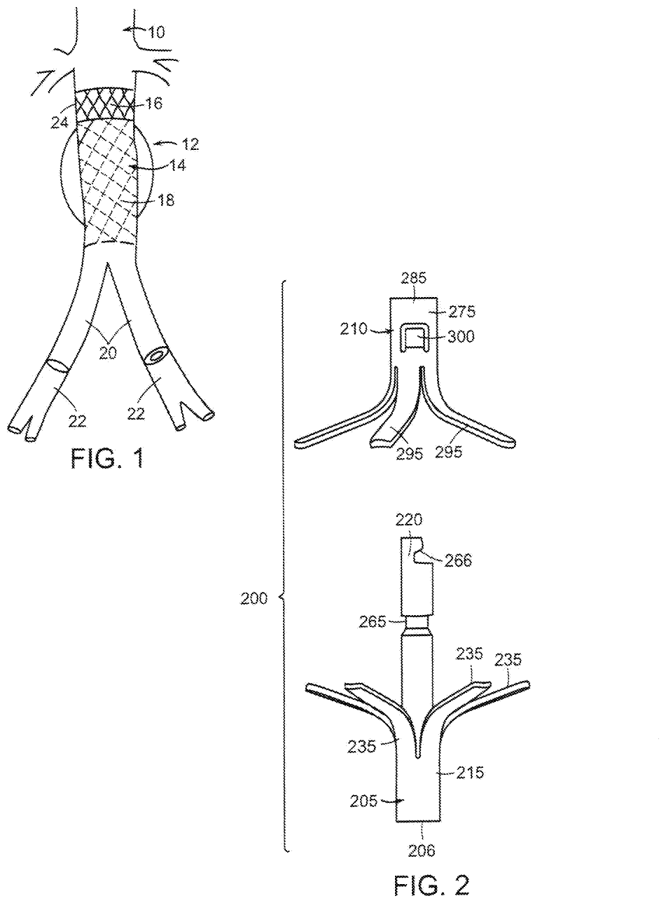

FIG. 1 illustrates, diagrammatically, a portion of an abdominal aorta 10 in which an aneurysm 12 has developed and through which a stent graft 14 has been placed to isolate and relieve the aneurysmal region from exposure to the patient's blood pressure. The stent-graft 14 includes a generally tubular stent in the form of a wire-like framework 16 to which a tubular graft 18 is attached. Typically, the graft 18 is formed from a woven, non-woven or knitted biocompatible fabric and may be bifurcated to have tubular segments 20 adapted to extend into the iliac arteries 22. The stent-graft 14 can be delivered and deployed by a catheter that can be introduced percutaneously into the patient's vasculature and advanced to the aneurysmal target site in the aorta. A number of such stent-graft catheters are available commercially and are in common use. Typically, the stent-graft is contained in the catheter in a contracted, radially collapsed, low profile configuration as the catheter is advanced to the aorta. When in position to be deployed, the stent-graft is released from the catheter and is expanded radially to engage the wall of the aorta. Typically, the stent-graft is deployed so that its upper portion is above the aneurysmal region where the stent-graft can engage with a healthy portion of the aortic wall as suggested at 24. When properly placed, the stent-graft 14 redefines the path of blood flow through the compromised portion of the aorta 12 to isolate the aneurysm from the patient's blood pressure.

Ideally, the engagement of the stent-graft 14 with a healthy portion of the aorta would maintain the stent graft in position. Continued exposure to pulsatile blood flow as well as pulsating expansion and contraction of the aortic wall, however, may, over time, cause the stent-graft to migrate downstream, possibly allowing blood to enter the space between the stent-graft and the weakened portion of the aortic wall, presenting a risk to the patient. In order to reduce that risk, it has become common to provide additional fixation means to secure the stent-graft in place. These have included, for example, incorporation of hooks or barbs into the stent framework that are intended to dig into the tissue of the aortic wall or the use of corkscrew-like fasteners to pierce the stent-graft and the aortic wall and hold the stent-graft in place. Such devices may tend to damage those pierced portions of the aorta and may result in the fixation devices working loose over time.

A fixation device that is less damaging to tissue is described in U.S. Pat. No. 10,076,339 and U.S. patent application Ser. No. 15/699,975, filed Sep. 8, 2017, the disclosures of which are hereby incorporated by reference, in their entireties. An embodiment of the fixation fastener is illustrated in FIG. 2 which shows the components of the fastener in exploded view. The fastener 200 comprises a distal implant 205 and a proximal implant 210 that cooperate to fasten layers of tissue or non-tissue together. Distal implant 205 comprises a tubular body 215 having a distal end 226. A locking tube 220 is located within the lumen of the body 215. The lower, distal end of the locking tube 220 is secured to the lower, distal end of the body 215 as by spot welds so that distal body and locking tube form a singular structure. The proximal end of the body 215 has a plurality of longitudinally extending slits that define a plurality of legs 235.

Distal implant body 215 preferably is formed out of an elastic material (e.g., a shape memory material having superelastic properties such as Nitinol or superelastic polymers) and constructed so that the legs 235 normally are bent and project laterally away from the longitudinal axis of the body 215 as shown in FIG. 2. By way of example, but not limitation, distal implant locking tube 220E may be formed out of a titanium alloy such as Ti5 AL-4V or Nitinol. Due to the elastic nature of the material used to form distal implant body 215, legs 235 can be deformed to a tubular, substantially linear, low profile shape so that they can be constrained within the lumen of a delivery tube or needle. However, when the constraint is removed, the elasticity of the material of the body 215 causes legs 235 to return to their relaxed, expanded position shown in FIG. 2.

FIG. 2 also illustrates the proximal implant 210 having a distal end 280, a proximal end 285, and a lumen adapted to receive the proximal end of the locking tube 220 of the distal implant 205. Tube 275 is slit at its distal end to define a plurality of legs 295. One or more inwardly projecting locking tangs 300 are formed in tube 275 adjacent its proximal end 285. Proximal implant 210 is preferably formed out of the same or similar material as the distal implant and is constructed so that its legs 295 normally project laterally away from the longitudinal axis of tube 275 as shown in FIG. 2. Legs 295 can be constrained inwardly to a low-profile configuration so that proximal implant 210 can assume a substantially linear disposition to be contained within the lumen of a delivery tube. However, when the constraint is removed, the elastic nature of the material causes legs 295 to return to the expanded position shown in FIG. 2. The distal implant 205 and proximal implant 210 are configured to lock together in a clamped position by cooperative engagement of one or more tangs 300 of proximal implant 210 and one or more tang-receptive circumferential grooves or recesses 265 on the locking tube 220.

In a preferred embodiment, the proximal and distal implants may be arranged so that when they are connected, legs of one are interdigitated with legs of the other, at least in the absence of tissue between the implants. Interdigitation refers to an arrangement that, when the proximal and distal implants are connected the legs 295 of the proximal implant will overlie the spaces between the legs 235 of the distal implant (or vice versa), as discussed in further detail below. With that arrangement, the legs of the fastener cooperate to apply oppositely directed forces at circumferentially alternating locations about the center of the fastener. We have found that application of clamping forces in this manner results in reduced risk of tissue damage while providing secure fixation and sealing of fastened layers of tissue or non-tissue. By applying clamping forces in this manner the clamped tissue and/or non-tissue layers can be constrained in a serpentine pattern that circumscribes the center of the fastener.

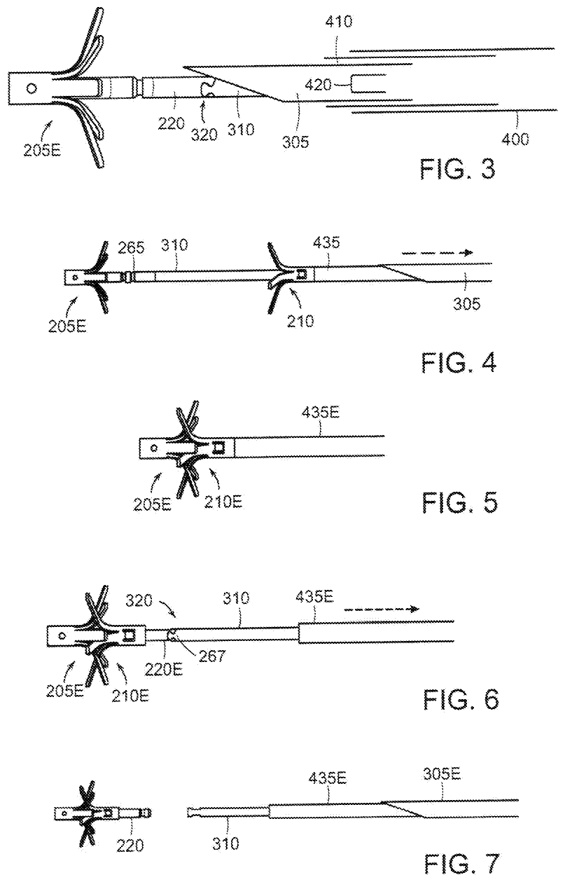

Additionally, the proximal end of the locking tube 220E may be provided with a first half 266 of a mechanical interlock 320 by which the locking tube 220 (and hence distal implant 205) can be connected to a distal implant retention tube 310 (FIGS. 3, 4, 6, 7) that has a mating second half 267 of the mechanical interlock 320 as described below. The distal implant retention tube 310 extends proximally to the proximal end of the catheter so that it can be manipulated by the clinician. The proximal implant is slidably disposed on the distal implant retention tube. The mechanical interlock enables the distal implant to remain attached to the deployment device until the proximal implant has been deployed and secured to the distal implant, as described below. The halves of the interlock 266 comprise a stepped configuration and are complementary so as to mate together. Although we have found that the connection tends to stay together, a locking rod (not shown) may be passed through the interlock 320 to further secure the connection. The rod must be removed or withdrawn before separation of the interlock components.

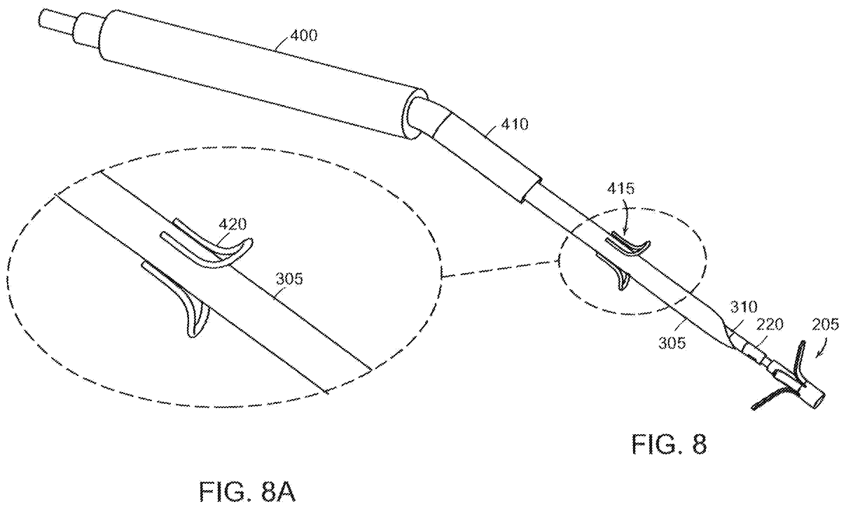

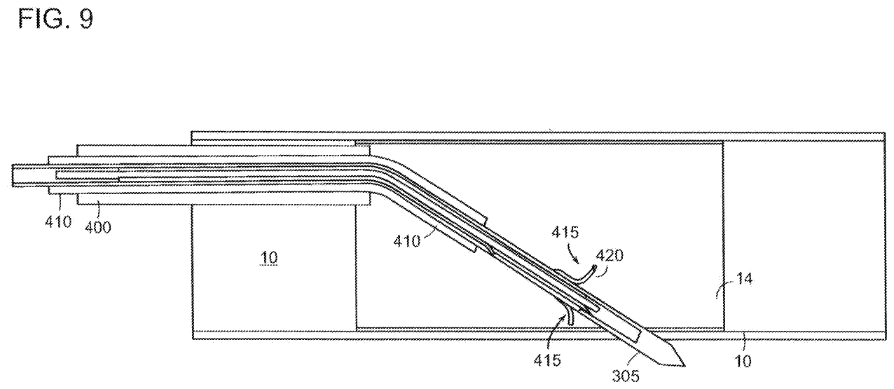

The manner in which the fastener 200 may be deployed is illustrated, somewhat diagrammatically, in FIGS. 3-7. The system includes an outer catheter 400 that contains a needle sleeve 410 that, in turn contains a needle 305 adapted to pierce the walls of the stent-graft and the aorta. The needle 305 is loaded with the distal and proximal implants in tandem and in their low-profile configuration in readiness to be deployed sequentially from the distal end of the needle. In order to deploy the fixation fastener, after the stent-graft has been deployed and the deployment apparatus has been withdrawn, the catheter 400 is advanced into the vasculature, using well-known techniques, and is navigated to the site of the stent-graft. The needle sleeve 410 containing the loaded needle 305 is advanced out of the delivery catheter (or the delivery catheter is retracted to expose the needle sleeve) (FIG. 3). The needle sleeve is constructed so that its distal end is normally biased in a bent configuration (FIG. 8) when freely outside of the constraint of the delivery catheter so as to be aimed in a somewhat radially outward direction. With the distal end of the needle sleeve directed toward a portion of the stent-graft that is positioned in healthy aortic tissue, the needle is advanced out of the needle sleeve (FIG. 3) toward and through the stent graft and the aorta to locate the needle outlet just beyond the outer surface of the aorta (FIG. 9).

The extent to which the tip of the needle protrudes beyond the aorta wall is limited in order to avoid potentially injuring tissues or organs adjacent to the fixation site. In order to so limit the needle penetration, the needle 305 is provided with one or more needle stops 415 mounted on the exterior of the needle a short distance behind the needle tip (FIGS. 8-9). The needle stop may take any number of forms, for example, as a resilient wire element 420 welded or otherwise secured to the needle. The stop should be formed from a material having elasticity to enable the stop to be folded flat against the exterior of the needle and held in that position by the needle sleeve 410 until the constraint of the needle sleeve is withdrawn, at which time the stop 415 can spring back to its normal position protruding radially of the needle (FIG. 8A). With the distal tip of the needle positioned outside of the aorta, the distal implant can be advanced out of the needle to a location outside the aorta where its legs self-expand to their deployed configuration (FIG. 3). Then, the needle and needle sleeve can be retracted within the catheter 400 to position the needle tip inside of the stent-graft where the proximal implant can be deployed within the stent-graft (FIG. 4). The proximal implant is deployed by a pusher tube 435 that is slidably disposed over the distal implant retention tube 310 and proximally of the proximal implant so that it can urge the proximal implant distally along the distal implant retention tube. With the proximal implant deployed inside of the stent-graft, the proximal and distal implants then can be manipulated by the retention tube and pusher tube to bring the implants together and be locked. The needle then can be retracted into the needle sleeve to urge the needle stops to their low-profile configuration, the retention tube and locking tube then can be disengaged and the delivery device can be removed.

It should be understood that the foregoing description is intended merely to be illustrative of the invention and that other modification, embodiments and equivalents may be apparent to those skilled in the art.

We Claim:

1. An apparatus for securing an endoluminal prostheses to the wall of an anatomical vessel comprising:
a delivery catheter having a distal end;
a needle having a sharp distal end and disposed in the catheter, the needle being extendable distally beyond the distal end of the catheter;
a fastener assembly contained in the needle, the fastener assembly having a distal implant and a proximal implant, the implants being advanceable separately and sequentially through and out of the needle;
the distal implant comprising a distal body, a first locking element, and a self-expandable portion adapted to self-expand from a diametrically-reduced delivery configuration to a diametrically-expanded deployed configuration;
the proximal implant comprising a proximal body, a second locking element, and an expandable portion adapted to self-expand from a diametrically-reduced delivery configuration to a diametrically-expanded deployed configuration;
means for directing the extended needle in a radial direction;
whereby, with the distal end of the catheter within the prosthesis, the needle can be advanced out of the catheter and be directed radially outwardly through both the prosthesis wall and the vessel wall, then to eject and deploy the distal implant out of the needle, then to withdraw the needle back into the prosthesis and to eject and deploy the proximal implant within the lumen of the prosthesis;
wherein the deployed implants are connectible together to fix the vessel and prosthesis together and to engage the first and second locking elements, thereby locking the distal implant and the proximal implant together;
the needle having a stop member disposed on its exterior to limit the extent to which the needle can protrude beyond the vessel wall.

2. The apparatus as defined in claim 1 wherein the stop member is movable between a radially extended position which it may function as a stop member and a low-profile, diametrically reduced position.

3. The apparatus as defined in claim 2 wherein the stop member is formed from an elastic material and wherein the stop member self-expands upon advancement of the needle out of the catheter and returns to its radially reduced position as the needle is retracted back into the catheter.

4. The apparatus as defined in claim 1 wherein the means for directing the needle in a radially outward direction comprises a needle sleeve movable within the catheter and slidably disposed over the needle, the sleeve having proximal and distal portions, the distal portion being disposed at an angle to the proximal portion when the distal portion of the sleeve is advanced out of the catheter.

5. The apparatus as defined in claim 3 further comprising:
a needle sleeve being movable relative to the needle between a position in which the stop member is constrained in its low-profile configuration by the needle sleeve and another position in which the stop member is free to assume its radially expanded position.

6. The apparatus as defined in claim 4 wherein the needle sleeve is formed from a resilient material that enables the proximal and distal portions of the sleeve to be withdrawn into the catheter and enable the distal portion to assume its angled position when extended from the catheter.

7. The apparatus as defined in claim 1 wherein the expandable portions of each of the implants comprise a plurality of radially extendable legs, and where the configurations of the legs of the connected implants are interdigitated

8. A method for fixing an endoluminal prosthesis to the wall of an anatomical vessel comprising:
providing the apparatus of claim 1;
advancing the catheter endoluminally through a patient's vasculature and into a previously placed endovascular prosthesis;
extending the needle from the catheter and advancing it radially to pierce the prosthesis and the vessel wall and limiting the extent to which the distal tip of the needle protrudes beyond the vessel wall;
deploying the distal implant from the needle externally of the vessel wall;
retracting the needle back into the prosthesis and then deploying the proximal implant;
drawing the implants together to fix the prosthesis to the vessel wall and lock the implants together with a portion of the fastener transfixing both the prosthesis and the vessel wall.
